(19) United States
(12) Patent Application Publication
(10) Pub. No.: US 2009/0068244 A1
(43) Pub. Date: Mar. 12, 2009

(54) Polymeric/carbon composite materials for use in medical devices

(75) Inventors: Jan Weber, Maastricht (NL); Thomas J. Holman, Princeton, MN (US); Graig L. Kveen, Maple Grove, MN (US)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc., Maple Grove, MN (US)

(21) Appl. No.: 11/900,779

(22) Filed: Sep. 12, 2007

(51) Int. Cl. A61L 27/54; A61L 27/44

(52) U.S. Cl. 424/423

(57) ABSTRACT

The invention provides implantable or insertable medical devices, which contain one or more composite regions. These composite regions, in turn, contain polymer and carbon particles. Also, the invention provides composite materials for use in a medical device containing styrene-isobutylene copolymer and carbon nanotubes.

### FIELD OF THE INVENTION

The present invention relates to new and improved materials for the construction of medical devices. In particular, the present invention relates to composite polymeric/carbon materials and medical devices which contain biocompatible copolymer materials and carbon particles, including devices having a composite region made of carbon particles and polymers, particularly styrene-isobutylene copolymers.

### BACKGROUND OF THE INVENTION

Polymer-based materials have been utilized for the construction of medical devices for many years. In particular, polymer materials, which deliver therapeutic agents to the body, have been the subject of intense interest. In accordance with some typical delivery strategies, a therapeutic agent is provided within a polymeric carrier layer and/or beneath a polymeric barrier layer that is associated with a medical device. Once the medical device is placed at the desired location within a patient, the therapeutic agent is released from the medical device at a rate that is dependent upon the nature of the polymeric carrier and/or barrier layer.

Materials which are suitable for use in making implantable or insertable medical devices typically exhibit one or more of the qualities of exceptional biocompatibility, extrudability, elasticity, moldability, good fiber forming properties, tensile strength, durability, and the like. Moreover, the physical and chemical characteristics of the device materials can play an important role in determining the final release rate of the therapeutic agent.

As a specific example, block copolymers of polyisobutylene and polystyrene, for example, polystyrene-polyisobutylene-polystyrene triblock copolymers (SIBS copolymers), which are described in U.S. Pat. No. 6,545,097 to Pinchuk et al., hereby incorporated by reference in its entirety, have proven valuable as release polymers in implantable or insertable drug-releasing medical devices. As described in Pinchuk et al., the release profile characteristics of therapeutic agents such as paclitaxel from SIBS copolymer systems demonstrate that these copolymers are effective drug delivery systems for providing therapeutic agents to sites in vivo.

These copolymers are particularly useful for medical device applications because of their excellent strength, biostability and biocompatibility, particularly within the vasculature. For example, SIBS copolymers exhibit high tensile strength, which frequently ranges from 2,000 to 4,000 psi or more, and resist cracking and other forms of degradation under typical in vivo conditions. Biocompatibility, including vascular compatibility, of these materials has been demonstrated by their tendency to provoke minimal adverse tissue reactions (e.g., as measured by reduced macrophage activity). In addition, these polymers are generally hemocompatible as demonstrated by their ability to minimize thrombotic occlusion of small vessels when applied as a coating on coronary stents. Despite these excellent properties, medical devices containing SIBS typically are not constructed from free standing films made of SIBS but rather, SIBS is provided as a coating or is integrated or incorporated into another material which forms the structure of the medical device.

Carbon-based materials have also been the subject of extensive research for biological applications. Carbon is an inert material and thus is generally naturally biocompatible. Structures made of carbon materials are being investigated as substrates for cell scaffolding and growth. For example, carbon nanotube (“CNT”) technology is being applied to medical applications, with recent investigations focusing on carbon nanotubes as substrates for the growth of retinal cells, neural cells and endothelial cells. See Correa-Duarte, “Fabrication and Biocompatibility of Carbon Nanotube-Based 3D Networks as Scaffolds for Cell Seeding and Growth,” Nanoletters, 4(11):2233-2236 (2004), the contents of which are incorporated by reference in their entirety. Also, CNT-based composites have been investigated for cartilage regeneration and in vitro cell proliferation of chondrocytes, and functionalized CNTs have been investigated for neuronal cell growth. Carbon nanotubes are strong, possess desirable electrical properties, and can be functionalized with a variety of molecules and are being explored in basic and applied medical research with the potential for a wide variety of medical applications. Certain CNTs are not only mechanically strong and electrically conductive, they are also capable of being shaped into 3D architectures and are promising in the construction of engineered products for biological applications.

There is a continuing need for novel materials for the construction of medical devices. In particular, it would be advantageous to provide materials that, in addition to the biocompatibility, biostability, and physical and chemical properties of known polymers such as SIBS, provide not only enhanced drug release properties but also enhanced mechanical and electrical characteristics such as that exhibited by carbon-based materials, including enhanced strength, rigidity, toughness and/or abrasion resistance and electrical conductivity. In addition, there is a continuing need for stable coatings for stents and other medical devices that support cell adhesion and proliferation.

These and other needs are addressed by the compositions, devices and techniques of the present invention.

### SUMMARY OF THE INVENTION

According to an aspect of the invention, implantable or insertable medical devices are provided, which contain or consist of one or more composite regions. These composite regions, in turn, contain polymers and carbon particles.

An advantage of the present invention is that medical devices can be provided with composite regions, which provide for enhanced mechanical characteristics, including enhanced strength, toughness and/or abrasion resistance and enhanced electrochemical and conductivity properties.

Another advantage of the present invention is that medical devices are provided that support cell adhesion and proliferation and otherwise support biological mechanisms.

Yet another advantage of the present invention is that medial devices and materials are provided that provide enhanced drug delivery of therapeutic agents to a target bodily site that possess the beneficial characteristics of both polymeric and inorganic materials.

These and other aspects, embodiments and advantages of the present invention will become immediately apparent to those of ordinary skill in the art upon review of the Detailed Description and Claims to follow.

### BRIEF DESCRIPTION OF THE DRAWINGS

FIG. 1 is a representation of films formed using (a) a two-layer approach wherein a layer of SIBS is drop cast followed by application of a single-wall carbon nanotube (“SWNT”) dispersion and (b) a one-layer approach wherein the SWNT is dispersed in a solution of SIBS in solvent and a film is cast from the resulting SWNT/SIBS dispersion.

FIG. 2 provides cross-sectional views of SIBS/CNT composites formed using separately formed films of SIBS and CNT.

FIG. 3 shows cross-sectional, expanded and side views of a stent assembly 20 that has been constructed having CNT film layers and a SIBS film layer.

FIG. 4 is a scanning electron micrograph (SEM) of a high surface area SIBS structure. The textured surface with replete with pore-like interstices.

FIGS. 5(a)-(e) are optical images of five CNT/biomolecule dispersions: SWNT-deoxyribonucleic acid (“SWNT-DNA”) (a), SWNT-Chondroitin (b) SWNT-Heparin (c) SWNT-Chitosan (“CH”) (d) and SWNT-Hyaluronic Acid (“HA”) (e).

FIG. 6 is a graphical representation showing the sedimentation of SWNT-DNA dispersion as a function of time. Sonication conditions were 35% for 45 min at 2 sec ON and 1 sec OFF.

FIG. 7 is a graphical representation of a cyclic voltammogram obtained for SWNT-DNA (40 μg) cast on 0.07 cm2 gas chromatography (“GC”) electrode in 0.2M phosphate-buffered saline solution (“PBS”) (pH 7.4), 50 mV/sec.

FIG. 8 is a graphical representation of a cyclic voltammogram obtained for DWNT-DNA (125 μg) on 0.07 cm2 GC electrode in 0.2M PBS (pH 7.4), 50 mV/sec.

FIG. 9 is a graphical representation of a cyclic voltammogram obtained for SWNT-DNA (40 μg) cast on 0.07 cm2 GC electrode in 1.0M NaCl.

FIGS. 10(a)-9(b) are fluorescence images of L929 mouse fibroblast cells cultured for 48 hours on DWNT/Chitosan coating on polypropylene (“PP”) (a) and polystyrene (b).

FIGS. 11(a)-10(b) are fluorescence images of L929 cells cultured for 48 hours on (a) DWNT/DNA/polystyrene and (b) PP.

FIGS. 12(a)-10(b) are fluorescence images of calcein-stained L929 cells cultured for 48 h on (a) DWNT/DNA and (b) DWNT/CH coating on polystyrene.

FIG. 13 is a light micrograph of (a) a single layer 0.15% SWNT/5% SIBS film and (b) a 2-layer 0.15% SWNT on 5% SIBS film both cast on glass.

FIGS. 14(a)-(b) are scanning electron micrographs (SEM) of (a) 5% SIBS film and (b) the same film (single layer) containing 0.15% SWNTs.

FIGS. 15(a)-(b) are SEMs of 0.15% SWNT film cast onto (a) stainless steel or onto (b) a pre-cast 5% SIBS layer.

FIG. 16 is a field-emission scanning electron microscopy image (“FESEM”) of drop cast mixed 5% SIBS (left image) and combined 5% SIBS and 0.15% SWNT layer (single layer film).

FIG. 17 is a FESEM of (a) drop cast 0.15% SWNT film and (b) drop cast two-layer film formed from preformed 5% SIBS layer coated by 0.15% SWNT layer.

FIG. 18 is a graphical representation of a cyclic voltammogram of 0.15% SWNT film on glass in 1 mM K₃Fe(CN)₆.

FIG. 19 is a graphical representation of a cyclic voltammogram of a single layer 0.15% SWNT and 5% SIBS coating on glass in 1 mM Fe(CN)₆⁴⁻.

FIG. 20 is a graphical representation of a cyclic voltammogram of a two-layer 0.15% SWNT on 5% SIBS coating on glass in 1 mM Fe(CN)₆⁴⁻.

FIG. 21 is a graphical representation of a cyclic voltammogram of 5% SIBS, 2 layer 0.25% SWNT on 5% SIBS and single layer 0.25% SWNT/5% SIBS coating on indium tin oxide (“ITO”)-glass in phosphate buffer.

FIGS. 22(a)-(b) are phase contrast micrographs of L929 cells growing on SIBS coatings on glass cover slips. (a) is a phase contrast microscopy image of cells only; (b) is a phase contrast microscopy image of cells containing MTT (“3-(4,5-dimethylthiazol-2-yl)-2,5-diphenyltetrazolium bromide”)-assay product.

FIG. 23 is a phase contrast micrograph of L929 cells growing on single layer 0.125% SWNT/SIBS coatings on glass cover slips and stained with MTT reagent.

FIG. 24 shows L929 cells growing on single layer 0.125% SWNT/SIBS coatings on glass cover slips and stained with Calcein AM. Cells were visualized using a combination of fluorescence and white light microscopy.

FIG. 25 is a graphical representation showing the relationship between corrected absorbance and seeded L929 cell number for the MTT assay.

FIG. 26 is a graphical representation showing the relationship between corrected absorbance and number of L929 cells seeded to 96-well PP plates and cultured for 48 hours (MTS assay).

FIG. 27 is a graphical representation of MTS assay results for SWNT and/or SIBS coatings on 96-well PP plate.

### DETAILED DESCRIPTION OF THE INVENTION

A more complete understanding of the present invention is available by reference to the following detailed description of numerous aspects and embodiments of the invention. The detailed description of the invention which follows is intended to illustrate but not limit the invention. The scope of the invention is defined by the claims.

In one aspect, the present invention provides implantable or insertable medical devices comprising one or more composite regions: composite carrier regions which contain polymers and carbon particles and/or composite barrier regions. In another aspect, the invention provides composite materials for use in a medical device comprising a composite region, said composite region comprising a composite carrier region comprising carbon particles and a polymer, wherein the polymer comprises a biocompatible polymeric material comprising styrene-isobutylene copolymer and the carbon particles comprise carbon nanotubes. In some embodiments, the composite carrier region comprises a first layer comprising a polymer and a second layer comprising carbon particles. In a further embodiment, these first and second layers each have a surface and at least a portion of each of the surfaces are bonded to each other by application of heat, pressure, or an adhesive. These layers can comprise films including a polymer film and a film comprised of carbon particles. In certain preferred embodiments, the carbon particles comprise carbon nanotubes and the polymer comprises a styrene-isobutylene block copolymer.

Among other benefits, the composite regions may provide, for example, a variety of enhanced mechanical characteristics, including enhanced strength, toughness and abrasion resistance, and enhanced electrical properties, such as electrical conductivity. In certain embodiments, the composite comprises biocompatible polymers and inorganic carbon materials having excellent strength, biostability and/or other properties that make them particularly well-suited for use in implantable or insertable medical devices.

Medical devices for use in conjunction with the present invention include a wide variety of implantable or insertable medical devices, which are implanted or inserted either for procedural uses or as implants. Examples include balloons, catheters (e.g., renal or vascular catheters such as balloon catheters), guide wires, filters (e.g., vena cava filters), stents (including coronary artery stents, peripheral vascular stents such as cerebral stents, urethral stents, ureteral stents, biliary stents, tracheal stents, gastrointestinal stents and esophageal stents), stent grafts, vascular grafts, vascular access ports, embolization devices including cerebral aneurysm filler coils (including Guglilmi detachable coils and metal coils), myocardial plugs, pacemaker leads, left ventricular assist hearts and pumps, total artificial hearts, heart valves, vascular valves, tissue bulking devices, sutures, suture anchors, anastomosis clips and rings, tissue staples and ligating clips at surgical sites, cannulae, metal wire ligatures, orthopedic prosthesis, joint prostheses, as well as various other medical devices that are adapted for implantation or insertion into the body.

The medical devices of the present invention include implantable and insertable medical devices that are used for diagnosis, for systemic treatment, or for the localized treatment of any tissue or organ. Non-limiting examples are tumors; organs including the heart, coronary and peripheral vascular system (referred to overall as “the vasculature”), the urogenital system, including kidneys, bladder, urethra, ureters, prostate, vagina, uterus and ovaries, eyes, lungs, trachea, esophagus, intestines, stomach, brain, liver and pancreas, skeletal muscle, smooth muscle, breast, dermal tissue, cartilage, tooth and bone. As used herein, “treatment” refers to the prevention of a disease or condition, the reduction or elimination of symptoms associated with a disease or condition, or the substantial or complete elimination of a disease or condition. Typical subjects (also referred to as “patients”) are vertebrate subjects, more typically mammalian subjects and even more typically human subjects.

In some embodiments, the composite regions correspond to entire medical devices. In other embodiments, the composite regions correspond to one or more medical device portions. For instance, the composite regions can be in the form of one or more strands which are incorporated into a medical device, in the form of one or more layers formed over all or only a portion of an underlying medical device substrate, and so forth. Layers can be provided over an underlying substrate in a variety of locations, and in a variety of shapes (e.g., in desired patterns), and they can be formed from a variety of composite materials (e.g., different composite compositions may be provided at different locations).

Materials for use as underlying medical device substrates include polymeric materials, both naturally-occurring (e.g., collagen) and synthetic (e.g., SIBS), ceramic materials and metallic materials, as well as other inorganic materials such as carbon- or silicon-based materials. As used herein, a “layer” of a given material is a region of that material whose thickness is small compared to both its length and width. As used herein a layer need not be planar, for example, taking on the contours of an underlying substrate. Layers can be discontinuous (e.g., patterned). Terms such as “film,” “layer” and “coating” may be used interchangeably herein.

In some embodiments of the invention, a therapeutic agent is disposed within or beneath the composite regions, in which cases the composite regions may be referred to as carrier regions or barrier regions.

By “composite carrier region” is meant a composite region which further comprises a therapeutic agent and from which the therapeutic agent is released. By “composite barrier region” is meant a composite region which is disposed between a source of therapeutic agent and a site of intended release, and which controls the rate at which therapeutic agent is released. For example, in some embodiments, the medical device consists of a composite barrier region that surrounds a source of therapeutic agent. In other embodiments, the composite barrier region is disposed over a source of therapeutic agent, which is in turn disposed over all or a portion of a medical device substrate.

As indicated above, the composite regions of the present invention contain a combination of polymers and carbon particles.

As used herein, “polymers” are molecules that contain one or more chains, each containing multiple copies of the same or differing constitutional units, commonly referred to as monomers. An example of a common polymer chain is polystyrene

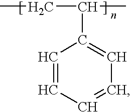

where n is an integer of 2 or more, typically 10 or more, 25 or more, 50 or more, 100 or more, 250 or more, 500 or more, or even 1000 or more, in which the chain contains styrene monomers:

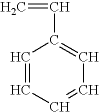

(i.e., the chain originates from, or has the appearance of originating from, the polymerization of styrene monomers, e.g., the addition polymerization of styrene monomers). In certain embodiments, the polymer within the composite region of the devices and compositions of the present invention comprises a biocompatible copolymer. In certain preferred embodiments, the polymer comprises a copolymer comprising a styrene-isobutylene copolymer. In yet other embodiments, the copolymer comprises a block copolymer comprising a polyisobutylene block and a polystyrene block, e.g., a polystyrene-polyisobutylene-polystyrene triblock copolymer.

Polymers for use in the composite regions of the present invention can have a variety of architectures, including cyclic, linear and branched architectures. Branched architectures include star-shaped architectures (e.g., architectures in which three or more chains emanate from a single branch point), comb architectures (e.g., architectures having a main chain and a plurality of side chains) and dendritic architectures (e.g., arborescent and hyperbranched polymers), among others. The polymers for use in the composite regions of the present invention can contain, for example, homopolymer chains, which contain multiple copies of a single constitutional unit, and/or copolymer chains, which contain multiple copies of at least two dissimilar constitutional units, which units may be present in any of a variety of distributions including random, statistical, gradient and periodic (e.g., alternating) distributions. Polymers containing two or more differing homopolymer or copolymer chains are referred to herein as “block copolymers.”

Polymers for use in the composite regions of the present invention may be selected, for example, from one or more of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-olefin copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097 to Pinchuk), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-, l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as further copolymers of the above.

The composite regions may comprise a wide range of polymer concentrations, ranging, for example, from about 1 wt % to 2 wt % to 5 wt % to 10 wt % to 25 wt % to 50 wt % to 75 wt % to 90 wt % to 95 wt % to about 99 wt % polymers.

By “carbon particles” is meant particles that are predominantly composed of carbon, typically containing about 75% to about 90 mol % to about 95 mol % to about 99 mol % or more carbon atoms. Carbon particles for use in the composite regions of the present invention may take on a variety of shapes, including spheres, polyhedra (e.g., fullerenes), solid cylinders (e.g., carbon fibers), tubes (e.g., carbon tubes, particularly single-wall carbon nanotubes, but also double-wall or multi-wall carbon nanotubes), plates (e.g., graphite sheets) as well as other regular and irregular shapes.

As used herein, carbon particles also include functionalized carbon nanotubes such as carboxylated SWNTs. Composite regions comprising functionalized carbon particles are within the scope of the present invention.

Purified SWNTs as well as functionalized carbon nanotubes are available commercially (e.g., Nanocyl, Belgium; NanoLab, Brighton, Mass.; CarboLex, Lexington, Ky.; Materials and Electrochemical Research Corporation, Tucson, Ariz., among a growing number of other suppliers). Non-covalent functionalization of carbon nanotubes has been the subject of great interest recently because it offers the potential to add a significant degree of functionalization to carbon nanotube surfaces (sidewalls) while still preserving nearly all of the nanotubes' intrinsic properties. For example, SWNTs can be solubilized in organic solvents and water by polymer wrapping (e.g., see e.g., Dalton et al., J. Phys. Chem. B. (2000) 104:10012-10016, the contents of which are incorporated by reference in their entirety) and nanotube surfaces can be non-covalently functionalized by adhesion of small molecules for protein immobilization (see e.g., Chen et al., J. Am. Chem. Soc. (2001) 123:3838-3839, the contents of which are incorporated by reference in their entirety). Materials and methods for preparing functionalized carbon nanotubes are disclosed in WO 2004/089819 A1, “Functionalized Carbon Nanotubes, A Process for Preparing the Same and Their Use in Medicinal Chemistry,” the contents of which are incorporated by reference in their entirety. See also Hu et al., “Chemically functionalized carbon nanotubes as substrates for neuronal growth,” Nano Letters, 4(3):507-511 (2004) and Mattson et al., “Molecular functionalization of carbon nanotubes and use as substrates for neuronal growth,” J. Mol. Neurosci., (June 2000) 14(3): 175-82, the contents of both of which are incorporated by reference in their entirety.

In addition to the various teachings for functionalizing carbon nanotubes, surface treatment additives for functionalizing carbon nanotubes are available commercially. For example, ZYVEX® (Richardson, Tex.) produces multi-functional surface treatments that non-covalently bridge carbon nanotubes to a polymer.

Carbon particles for use in the invention may vary widely in size. In many embodiments, their smallest dimensions (e.g., the thickness for plates, the diameter for spheres, regular polyhedrons, fibers and tubes, etc.) are less than 10 micrometers (e.g., ranging from 0.05 nm to 1 nm to 10 nm to 100 nm to 1 micrometer to 10 micrometers), whereas additional dimensions (e.g., the width for plates, and the length for fibers and tubes) may be of the same order of magnitude or much larger (e.g., ranging from 0.05 nm to 1 nm to 10 nm to 100 nm to 1 micrometer to 10 micrometers to 100 micrometers to 1000 micrometers or even more).

Preferred carbon particles are those that comprise molecular carbon that is predominantly in sp² hybridized form (i.e., structures in which the carbons atoms are predominantly connected to three other carbon atoms within a lattice structure, sometimes referred to as a “grapheme carbon lattice”). Examples of carbon particles that predominantly comprise carbon in sp² hybridized form include graphite, fullerenes (also called “buckyballs”) and carbon nanotubes. Graphite molecules contain planar sheets of sp² hybridized carbon, whereas fullerenes and carbon nanotubes contain curved sheets of sp² hybridized carbon in the form of hollow spheres and tubes, respectively. Fullerenes and carbon nanotubes may be thought of as sheets of graphite that are shaped into polyhedra and tubes and, in fact, may be made by directing a laser at a graphite surface, causing some of the sheets to be displaced from the graphite, which subsequently react to form fullerenes and/or nanotubes.

The composite regions may comprise a wide range of carbon particle concentrations, ranging, for example, from about 1 wt % to 2 wt % to 5 wt % to 10 wt % to 25 wt % to 50 wt % to 75 wt % to 90 wt % to 95 wt % to about 99 wt %.

In certain embodiments of the invention, the carbon particles are carbon nanotubes. Examples of carbon nanotubes include single-wall carbon nanotubes and multi-wall carbon nanotubes (which term embraces so-called “few-wall” carbon nanotubes). Specific examples of nanotubes include single wall carbon nanotubes (SWNTs), which have inner diameters ranging from 0.25 nanometer to 5 nanometers, and lengths up to 100 micrometers), double-wall nanotubes (DWNTs) and multi-wall carbon nanotubes (MWNTs), which have inner diameters ranging from 2.5 nanometers to 10 nanometers, outer diameters of 5 nanometers to 50 nanometers, and lengths up to 100 micrometers.

SWNTs are particularly preferred for many embodiments of the present invention. At present, the purest SWNTs are produced by pulsed laser vaporization of carbon that contains metal catalysts such as nickel and cobalt. Fullerenes are known to form when the carbon is vaporized, mixes with an inert gas, and then slowly condenses. The presence of a metal catalyst, however, is known to form SWNTs. SWNTs are generally considered to be individual molecules, yet as noted above, they may grow to be microns in length. SWNTs may also be produced by other processes such as arc discharge processes.

Regardless of the production technique, after formation, SWNTs are typically purified to remove impurities such as amorphous carbon and residual metal catalysts, for example, by exposure to NHO₃, followed by rinsing, drying, and subsequent oxidation at high temperatures. A specific technique for providing SWNTs with >99.98 wt % purity (as measured by inductively coupled plasma (“ICP”) analysis) is described in the Oak Ridge National Laboratory, Laboratory Directed Research and Development Program, Fy 2003, Annual Report. SWNTs are also commercially available as aqueous suspensions.

In some embodiments, the composite region may also comprise particles in addition to carbon particles, including various irregular and regular particles such as fibers, tubes, spheres, polyhedrons, plates, and so forth. Examples of particles that may be combined with the carbon particles in the composite regions of the invention include, for example, ceramic particles, such as alumina, titanium oxide, tungsten oxide, tantalum oxide and zirconium oxide particles, silica particles, and silicate particles including monomeric silicates and polyhedral oligomeric silsequioxanes (POSS).

As noted above, the medical devices of the present invention optionally contain one or more therapeutic agents. “Therapeutic agents,” “drugs,” “pharmaceutically active agents,” “pharmaceutically active materials,” and other related terms may be used interchangeably herein. These terms include genetic therapeutic agents, non-genetic therapeutic agents, cells and biologically active molecules. A wide variety of therapeutic agents can be employed in conjunction with the present invention including those used for the treatment of a wide variety of diseases and conditions (i.e., the prevention of a disease or condition, the reduction or elimination of symptoms associated with a disease or condition, or the substantial or complete elimination of a disease or condition). Numerous therapeutic agents are described here.

Exemplary therapeutic agents for use in conjunction with the present invention include the following: (a) anti-thrombotic agents such as heparin, heparin derivatives, urokinase, clopidogrel, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) alpha receptor antagonist (such as doxazosin, Tamsulosin) and beta receptor agonists (such as dobutamine, salmeterol), beta receptor antagonist (such as atenolol, metaprolol, butoxamine), angiotensin-II receptor antagonists (such as losartan, valsartan, irbesartan, candesartan and telmisartan), and antispasmodic drugs (such as oxybutynin chloride, flavoxate, tolterodine, hyoscyamine sulfate, diclomine) (u) bARKct inhibitors, (v) phospholamban inhibitors, (w) Serca 2 gene/protein, (x) immune response modifiers including aminoquizolines, for instance, imidazoquinolines such as resiquimod and imiquimod, and (y) human apolioproteins (e.g., AI, AII, AIII, AIV, AV, etc.).

Numerous therapeutic agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), rapamycin (sirolimus) and its analogs (e.g., everolimus, tacrolimus, zotarolimus, etc.), cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Particularly beneficial therapeutic agents include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, zotarolimus, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well as derivatives of the foregoing, among others.

In certain preferred embodiments, the therapeutic agent is an anti-proliferative agent comprising paclitaxel.

A wide range of therapeutic agent loadings can be used in connection with the medical devices of the present invention, with the therapeutically effective amount being readily determined by those of ordinary skill in the art and ultimately depending, for example, upon the condition to be treated, the age, sex and condition of the patient, the nature of the therapeutic agent, the nature of the composite region(s), the nature of the medical device, and so forth. Exemplary loadings range, for example, from 1 wt % or less to 2 wt % to 5 wt % to 10 wt % to 25 wt % or more of the composite region.

Numerous techniques are available for providing the composite regions for the medical devices in accordance with the present invention.

In many preferred embodiments, solvent-based techniques can be used to form the composite regions of the present invention, including solvent casting techniques, spin coating techniques, web coating techniques, solvent spraying techniques, dipping techniques, techniques involving coating via mechanical suspension including air suspension, ink jet techniques, electrostatic techniques, and combinations of these processes.

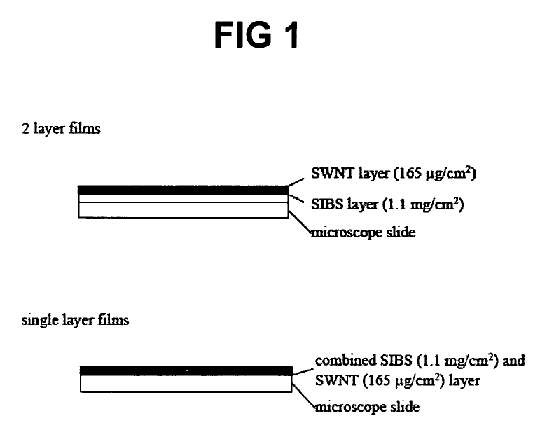

In certain preferred embodiments, polymer and carbon particle films that form the composite region are made using the following methods: 1) a two-layer approach wherein a layer of polymer, e.g., SIBS is drop cast and allowed to dry, followed by the application of a carbon particle dispersion, e.g., SWNT dispersion that is prepared from the same solvent as the initial SIBS layer, e.g. toluene, chloroform, tetrahydrofuran (THF) or cyclohexane; 2) a one-layer approach wherein the carbon particles, e.g., SWNT, is dispersed in a solution of SIBS in solvent and a film is cast from the resulting SWNT/SIBS dispersion. FIG. 1 shows representations of films formed using (a) a two-layer approach wherein a layer of SIBS is drop cast followed by application of a SWNT dispersion and (b) a one-layer approach wherein the SWNT is dispersed in a solution of SIBS in solvent and a film is cast from the resulting SWNT/SIBS dispersion. In both of these methods, the dispersion comprising carbon nanotubes and a solvent can optionally contain a surfactant or other surface treatment additive or chemical modifier.

Applicants have discovered that SIBS itself also acts as a dispersant on CNTs. As detailed below in the Examples, there were fewer occlusions when SIBS was included as a dispersant (in single layer films) indicating that the dispersion was assisted by SIBS. For SWNTs alone, drop dispersions were not as even, with thick and thin areas clearly visible, indicating that in the absence of SIBS, CNTs had coalesced upon drying of the films. This illustrated the improvements in coatings made in the presence of SIBS, either when incorporated in the dispersion, or when used as a base for casting films.

Where the composite regions are formed from one or more polymers having thermoplastic characteristics, then a variety of thermoplastic processing techniques may be used to form the polymeric release regions, including compression molding, injection molding, melt dispersion, blow molding, spinning, vacuum forming and calendaring, as well as extrusion into sheets, fibers, rods, tubes and other cross-sectional profiles of various lengths. Using these and other thermoplastic processing techniques, entire medical articles of portions thereof can be made.

In some embodiments of the invention, a polymer dispersion (where solvent-based processing is employed) or a polymer melt (where thermoplastic processing is employed) is applied to a substrate to form a composite region. For example, the substrate can correspond to all or a portion of an implantable or insertable medical device to which a composite region is applied. The substrate can also be, for example, a template, such as a mold, from which the composite region is removed after solidification. In other embodiments, for example, extrusion and co-extrusion techniques, one or more composite regions are formed without the aid of a substrate.

In certain embodiments, a therapeutic agent is disposed within at least one of the layers or films of the composite region. They can be embedded, disposed, incorporated or dissolved within a composite carrier region. If it is desired to provide one or more therapeutic agents and/or any other optional agents in the composite region, and so long as these agents are stable under processing conditions, then they can be provided within the dispersion or polymer melt and co-processed along with the composite region.

Alternatively, therapeutic and/or other optional agents may be introduced subsequent to the formation of the composite region. For instance, in some embodiments, the therapeutic and/or other optional agents are dissolved or dispersed within a solvent, and the resulting dispersion contacted with a previously formed composite region (e.g., using one or more of the application techniques described above, such as dipping, spraying, etc.).

As noted above, barrier layers are formed over a therapeutic-agent-containing region in some embodiments of the invention. In these embodiments, a composite barrier region can be formed over a therapeutic-agent-containing region, for example, using one of the solvent-based or thermoplastic techniques described above. Alternatively, a previously formed composite region can be applied over (e.g., by adhesion) a therapeutic agent containing region.

In other embodiments, the polymer film and the CNT films are separately formed and brought together to form the composite region. The composite region contains a first layer comprising a polymer film having a surface at least a portion of which surface is attached to the second layer by applying the surface with a solution comprising the polymer dissolved in a solvent. The solution is sprayed or applied to the surface of the first layer and then the first layer is bonded to the second layer.

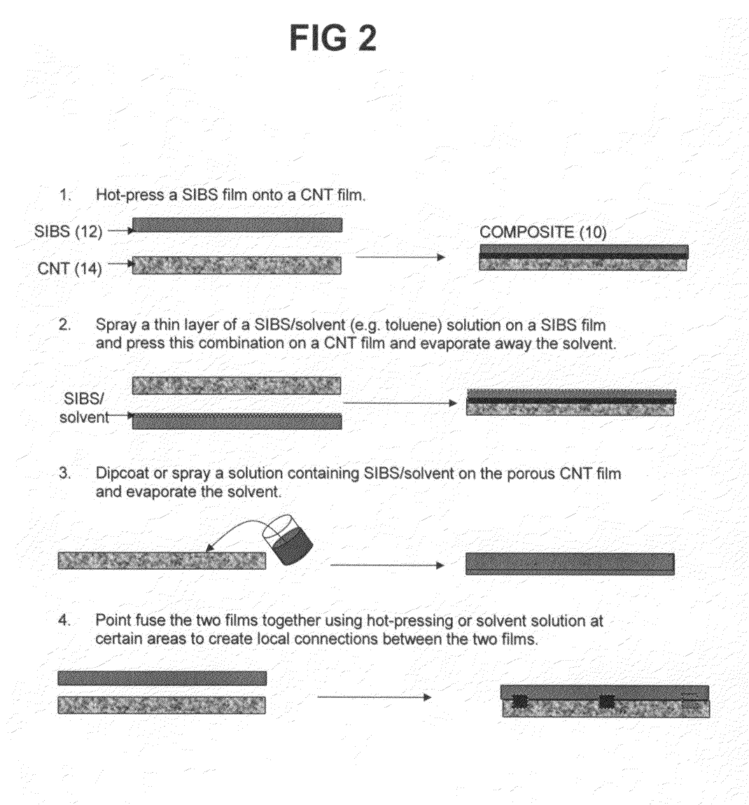

FIG. 2 provides cross-sectional views of a composite region 10 having a first layer comprising a SIBS film 12 and a second layer comprising a CNT film 14 that is formed in four ways. CNT films for the composite regions of the present invention can be readily prepared using commercially obtained SWNT dispersions (in Triton X-100 or toluene solution). Detailed instructions and protocols for the preparation of CNT films is provided in Weber et al., U.S. Pub. No. 2005/0074479 A1 and Rinzler et al., “Large scale purification of single-wall carbon nanotubes: process, product and characterization,” Applied Physics, A A67, 2937 (1998), the contents of both of which are incorporated by reference in their entirety.

SIBS film can be prepared following the disclosures of Pinchuk et al., U.S. Pat. No. 6,545,097. SIBS can be continuous, textured and discontinuous or perforated with holes. The SIBS film and the CNT film can be attached to one another through any variety of means known to one of skill in the art, including but not limited to exposure to heat (e.g., hot-pressing), pressure, or by the use of adhering agents. In some embodiments, the SIBS film can be adhered to the CNT film by using an “adhesive” solution 16 containing SIBS copolymer which is dissolved in a solvent base (e.g., toluene) and spraying, dipcoating, or otherwise applying a layer of the solution to the SIBS film or CNT film and contacting the surfaces of these films together which then bond to form the composite film.

As would be appreciated by one of skill in the art, any suitable bonding material or agent for attaching the layers to one another can be used. In other embodiments, the SIBS film and the CNT film may be attached only at certain adhesion points 18, for example, by point fusing the two films together to create a local connection without submitting the entire surfaces of CNT film or SIBS film to either heat treatment, or a solvent/polymer solution.

In some preferred embodiments, the carbon particles comprise carbon nanotubes and the polymer comprises a styrene-isobutylene block copolymer and the composite region comprises two or more layers with at least one layer comprising SIBS and at least one layer comprising carbon nanotubes. A variety of two- or multi-layer composite regions can be created that have any number of combinations of SIBS and CNT films having composite layer configurations such as A-B, A-B-A, A-B-A-B, B-A-B, etc., wherein A is a SIBS film and B is a CNT film. Therapeutic agents, such as biologically active molecules, and/or other optional agents may be present in any one or more of the various layers of the composite regions. For example, the SIBS film of FIG. 1 can be loaded with a therapeutic agent, e.g., paclitaxel.

In other preferred embodiments, the medical device comprises a stent having two ends and an interior surface and an exterior surface and either the first or second layer is disposed on at least a portion of the interior surface of the stent and either the first or second layer is disposed on at least a portion of the exterior surface. In some embodiments, the first layer covers the entire exterior surface of the stent and the second layer covers the entire interior surface of the stent. The first and second layers each have a surface and at least a portion of each of these surfaces are bonded to each other by application of heat, pressure, or an adhesive adjacent to the ends of the stent.

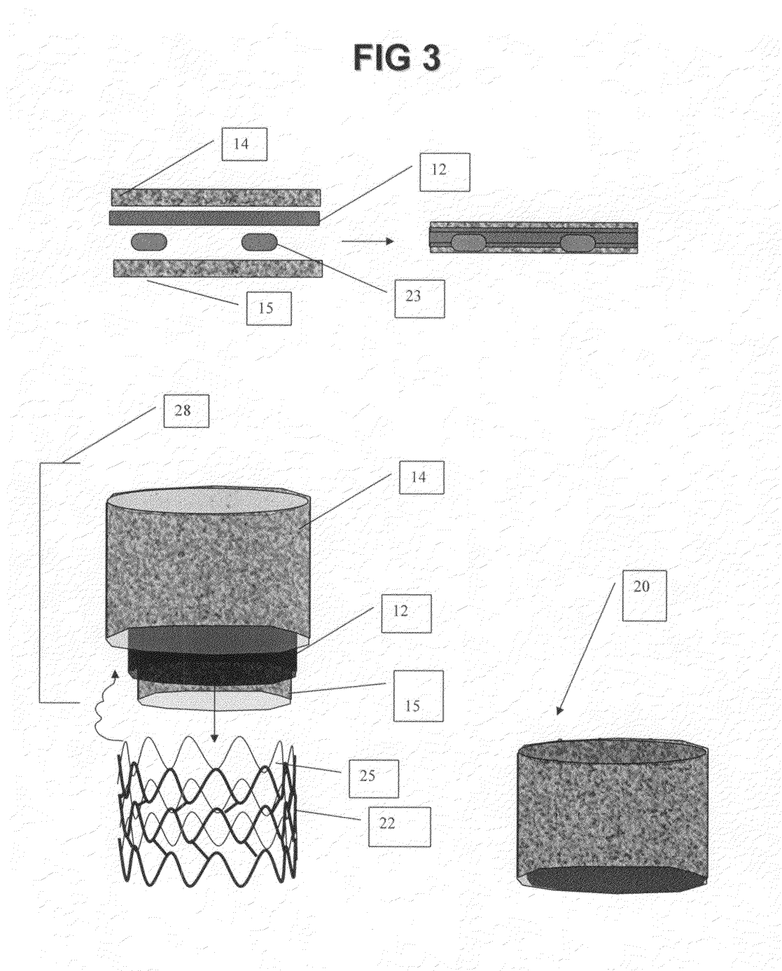

In some preferred embodiments, the present invention provides a composite material for use in an insertable or implantable medical device comprising a composite region made of at least one layer of carbon particles disposed over all or a portion of the device. Also, at least one layer of a polymer is disposed over all or a portion of the device and the polymer comprises a styrene-isobutylene copolymer and a therapeutic agent is disposed within the polymer. This embodiment is illustrated, for example, in FIG. 3.

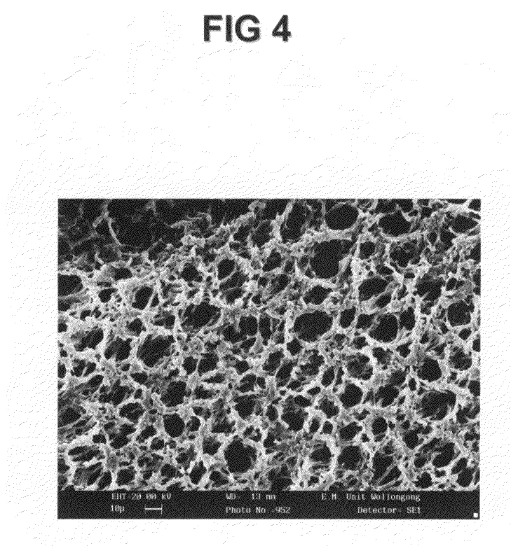

FIG. 3 shows cross-sectional, expanded and side views of a stent assembly 20 according to one embodiment of the invention that has been constructed having a CNT film layer 14, a SIBS film layer 12 that is loaded with a therapeutic agent, a second CNT film layer 15 and a metal or polymer stent body 22 having stent struts 23. The CNT film, in some embodiments, is porous and thus a therapeutic agent contained within the SIBS film can migrate through the various layers of the stent assembly 20. In some embodiments, the stent assembly 20 can itself be porous if the SIBS film 12 contains pores or is perforated as exemplified in the scanning electron micrograph image of a high surface area SIBS film surface shown in FIG. 4.

In other embodiments, the SIBS film forms a continuous layer and thus, not all of the layers of the composite region 28 within the medical device are porous. The porosity of the various components of the composite region can thus be modulated depending on the particular medical or biological application to achieve a specific level of bioactivity, drug delivery, cell adhesion or scaffolding properties, or mechanical properties. In FIG. 3, the stent 20 is comprised of numerous stent struts 23. The stent body 22 and the stent struts 23 are surrounded by various layers of the composite region 28, in this case a SIBS film layer 12 and two CNT layers 14, 15 to form a continuous wrapping around the stent body 22. In a separate embodiment, unlike the continuous wrapping shown in FIG. 3, only discrete portions of the stent body 22 and/or stent struts 23 are surrounded by any one or combination of the SIBS film layer and one or two CNT layers. The composite region 28 of this embodiment extends past both ends of the stent body 22 and covers not only the stent struts 23 but also the spaces 25 between the stent struts 23. The various layers of the composite region 28 can be coupled to the stent 22 through various methods and techniques. These techniques include mechanically attaching layers of the composite region 28 to the stent 22 by clamping, sewing, gluing or otherwise adhering the composite region 28 to the stent 22, forming the composite region 28 around the stent 22, or directly depositing the composite region 28 onto the stent 22, or functionalizing the stent 22 surface so that it forms a non-covalent or covalent bond with molecules of the composite region 28.

### EXAMPLE 1

### Preparation and Characterization of CNT/Biomolecule Dispersions and CNT/Biomolecule Films

Three CNT types (single wall carbon nanotube, multi wall carbon nanotube and double wall carbon nanotube) and four biomolecules (chondroitin sulfate, heparin (500,000 unit size), hyaluronic acid, and chitosan (water soluble)) were prepared into CNT-biomolecule dispersions and characterized.

Optical microscopy, particle sizing and Raman spectroscopy results indicate that DNA, chitosan and chondroitin act as very efficient dispersing agents. By combining these three techniques it was possible to evaluate the quality of the CNT biodispersions. The biomolecule dispersions that showed the most promise as coating materials were: SWNT-Chondroitin, SWNT-Hyaluronic acid and SWNT-Chitosan. The SWNT-DNA and SWNT-Chondroitin dispersions produced films on the stainless steel coupons that were stable upon immersion into electrolyte solutions. To promote good adhesion between the cast film and the stainless steel coupon it was necessary to UV treat the coupons for 20 min. The CNT biodispersions were suitable substrates for culture of L929 cells (mouse fibroblast cells, originally sourced from American Type Culture Collection (“ATCC”) Manassas, Va., obtained from Prof. Mark Wilson (Biological Sciences, University of Wollongong)). Preliminary studies show that DWNT/CH coatings were stable when coated onto tissue culture plastic ware.

### Preparation of CNT/Biomolecule Dispersions

1:1 weight ratio mixes of CNTs and biomolecules were mixed together in a round bottom vessel and sonicated (30% 2 sec ON, 1 sec OFF) for 45 min at room temperature. These dispersions were characterized using light microscopy, Raman spectroscopy (radial breathing bode (“RBM”) study), and particle size analysis. All of the results obtained were compared against SWNT-DNA dispersion data as DNA is known to be an extremely good dispersion agent.

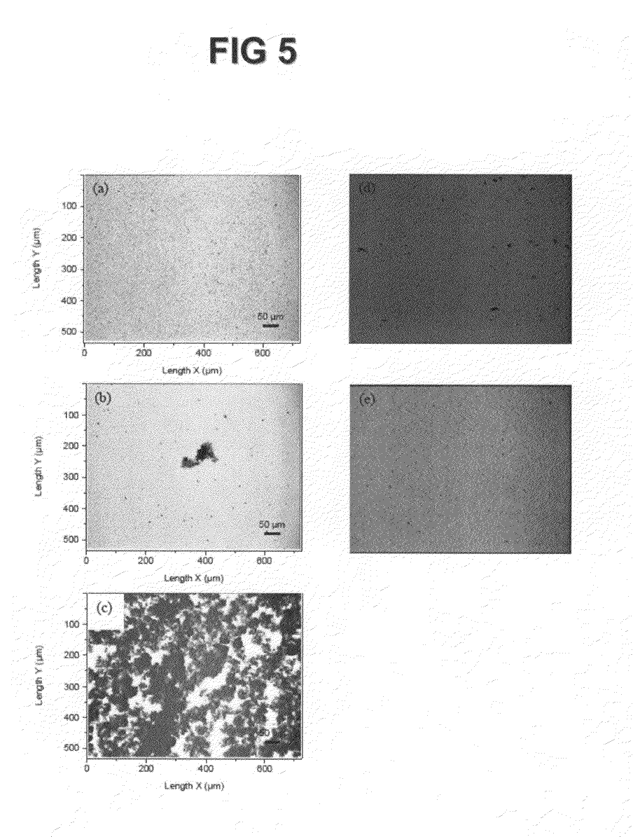

After sonication, all CNT/biomolecule dispersions (including the standard SWNT-DNA) appeared black and homogenous; however after 5 min, the CNT-heparin dispersion clearly separated into two phases. This indicated that heparin at 500,000 unit size in a poor dispersant for the CNT used. All other dispersions were stable after 65 days post formation with no visible separation occurring. Optical images of the dispersions are shown in FIG. 5(a)-5(e): (a) SWNT-DNA, (b) SWNT-Chondroitin, (c) SWNT-heparin, (d) SWNT-Chitosan, and (e) SWNT-Hyaluronic Acid. Weight percent ratio of CNT to biomolecule is 1:1. Sonication conditions were 30% for 45 min at pulsed sonication 2 sec ON and 1 sec OFF.

The chondroitin dispersion appeared to separate into two phases upon formation of the thin film used for optical analysis. These phases comprised of aggregations in the dispersion and the homogeneous phase surrounding the aggregates (FIG. 5(b)). Particle size analysis allowed for the sedimentation profiles to be plotted for each CNT biodispersion.

The sedimentation profile is indicative of the dispersive stability since an unstable dispersion will show phase separation and a large variation in particle size. Dispersions that are homogenously dispersed will show a narrow particle size distribution due to the lack of CNT aggregation. The size distribution for the SWNT-DNA, SWNT Chondroitin, SWNT-Heparin, SWNT-Hyaluronic Acid, and SWNT-Chitosan dispersions are shown in Table 1.

TABLE 1
Average particle size of the biodispersion
(by number average) after sonication.

| | | | Size |
|---|---|---|---|
| | | | Distribution |
| | Z average (nm) | % by Number | (nm) |
| SWNT-DNA | 208.4 | 99 | ~150 |
| SWNT-Chondroitin | 320.8 | 99 | ~300 |
| SWNT-Heparin | 104.0 | 99 | ~250 |
| SWNT-Hyaluronic Acid | 81.3 | 99 | ~200 |
| SWNT-Chitosan | 38.0 | 99 | ~220 |

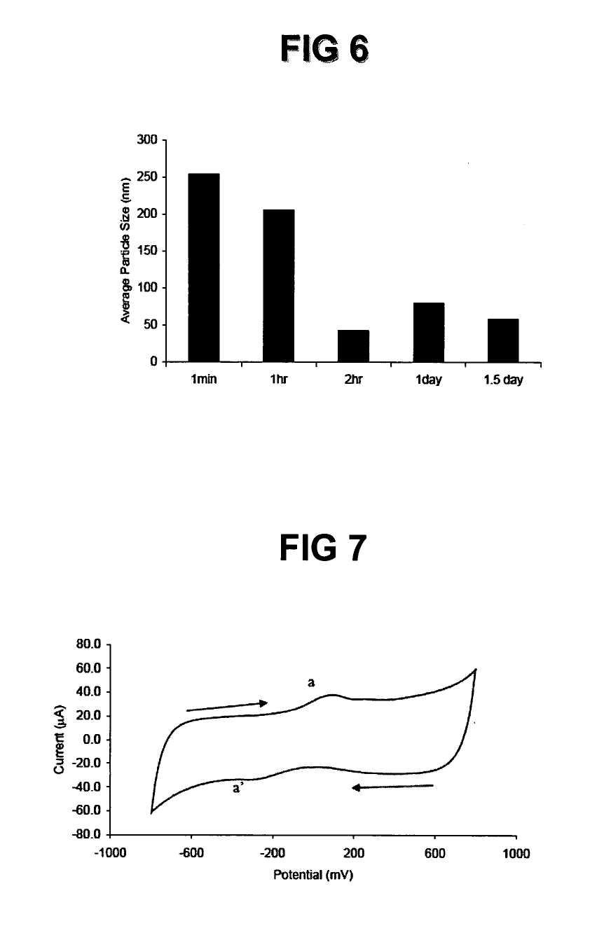

The plot in FIG. 6 shows the average particle size for the SWNT-DNA dispersion as a function of sedimentation time. It shows that by 2 h the dispersion was stable with an average particle size of 58 nm being recorded. The larger particle sizes observed at 1 min to 1 hour were assumed to have settled to the bottom of the cuvette. Upon investigation, there was a black deposit coating the bottom of the cuvette. The sedimentation profiles for the SWNT-Chondroitin, SWNT-Hyaluronic acid, SWNT-Chitosan and SWNT-Heparin showed a similar trend. However, the time it took for the particle size to stabilize was less. This is attributed to the larger size aggregates in solution depositing at the bottom of the cuvette. The particle size distribution of the stabilized dispersions was also larger than that of the SWNT-DNA dispersion (see Table 1). This may account for the smaller Z average particle size shown in Table 1. If these dispersions contained larger particles, which settled at a faster rate, than that of the SWNT-DNA dispersion, the particle sizing would have been performed on a solution which contained a lower amount of SWNTs which were present as smaller bundles.

Raman spectroscopy studies showed that the wavenumber shift, with respect to pristine SWNTs, in the radial breathing mode (RBM) of the SWNT dispersed in DNA, chitosan and hyaluronic acid is indicative of significant CNT-biomolecule interaction. Some interaction was also evident with chondroitin. The shift in wavenumber equates to an increase in energy required to induce resonance in the CNTs. The increase in energy is required due to the non-covalent functionalization of the CNTs by the biomolecules. The dispersions containing heparin showed very little wavenumber shift upon dispersing, suggesting no significant CNT heparin interaction (Table 2).

TABLE 2
Wavenumber and (wavenumber shift) of the RBM for the
SWNT dispersion Raman spectra. The shift is measured
against the RBM wavenumbers for the pristine SWNT.

Wavenumber (cm⁻¹)

| Pristine SWNT | 195.69 | 216.46 |
|---|---|---|
| SWNT-DNA | 200.45 (4.76) | 221.21 (4.75) |
| SWNT-Chondroitin | 197.46 (1.77) | 218.36 (1.9) |
| SWNT-Heparin | 195.93 (0.24) | 216.69 (0.23) |
| SWNT-Chitosan | 198.24 (2.55) | 219.18 (3.17) |
| SWNT-Hyaluronic acid | 198.24 (2.55) | 219.18 (3.17) |

### Formation of CNT-Chitosan-Heparin Composite Films

A preliminary study aimed at forming SWNT-Chitosan-Heparin composites using the layer by layer technique was carried out. It was found that heparin does not form stable, well dispersed CNT solutions, while chitosan is an excellent dispersant. Therefore, we attempted to utilize the known interaction between chitosan and heparin at pH 4.5 to make stable CNT-Chitosan-Heparin films. At pH 4.5, heparin carries a negative charge while chitosan is positively charged. The substrate used in this preliminary study was glass. We attempted to quantify the heparin content using the toluidine blue assay. This assay relies on toluidine blue complexing with heparin to vary the absorbance intensity of the toluidine blue at 629 nm.

### Soaking SWNT-Chitosan Films in Heparin Solution

The dispersion formulations were as follows:

1) Dispersion: 0.5% SWNT (50 mg)+0.5% Chitosan B (50 mg) in 10 ml H₂O at pH 4.5; and
2) Dispersion 2: 0.5% SWNT (50 mg)+0.5% Triton X-100 (50 mg) in 10 ml H₂O (blank).

The heparin solution was 1000 ppm (500,000 unit size), adjusted to pH 4.5 with 1.0 M HCl.

The composite films were prepared by taking 20 μl of SWNT-Chitosan or SWNT-Triton X-100 dispersion and casting onto glass slides and allowing them to dry. Each film was placed in the heparin solution for a period of time. Soaking time varied from 30 minutes to 5 hours. The glass slides were removed after the required time and dried. The UV absorption of heparin solution at 629 nm was measured using toluidine blue assay before and after soaking in SWNT-Chitosan and SWNT-Triton films.

Results are shown in Table 1.

TABLE 1
The uptake of heparin on the SWNT-Chitosan
film after different soak times.

| Soak time | | Heparin | Uptake of |
|---|---|---|---|
| (h) | Abs | (μg) | Heparin (%) |
| 0.5 | 0.146 | 50.30 | 3.18 |
| 1 | 0.144 | 50.66 | 2.50 |
| 2 | 0.140 | 51.38 | 1.12 |
| 3 | 0.139 | 51.55 | 0.079 |
| 4 | 0.141 | 51.96 | 0 |
| 5 | 0.140 | 51.38 | 1.12 |

### Layer-by-Layer (“LbL”) Deposition of SWNT-Chitosan on SWNT-Chitosan B Films

Up to 3 layers of SWNT-Chitosan B were sequentially deposited and dried on glass slides. The dried films were dipped up to 3 times in 2 ml of 1000 ppm heparin solution. The UV absorption of the heparin solution at 630 nm was measured using toluidine blue assay before and after dipping with SWNT-Chitosan film and layer-by-layer (“LbL”) deposition of SWNT-Chitosan B films in order to detect the loss of heparin from solution as a result of adsorption to SWNT-Chitosan B films. The results are shown in Table 2.

TABLE 2
The uptake of heparin on the SWNT-Chitosan
B film and LBL SWNT-Chitosan B films.

| Deposition | | | |
|---|---|---|---|
| SWNT-Chitosan | | Heparin | Uptake of |
| layer | Abs | (μg) | Heparin (%) |
| no SWNT-Ch | 0.146 | 50.30 | 3.18 |
| single layer-film | 0.148 | 49.95 | 3.58 |
| dipped once | 0.159 | 47.98 | 7.66 |
| 2 layer-film | 0.147 | 50.13 | 3.51 |
| dipped once | 0.157 | 48.34 | 6.97 |
| dipped twice | 0.143 | 50.84 | 2.16 |
| 3 layer-film | 0.143 | 50.84 | 2.16 |
| dipped once | 0.150 | 49.59 | 4.56 |
| dipped twice | 0.145 | 50.48 | 2.85 |
| dipped 3 times | 0.149 | 49.76 | 4.23 |

### Measurement of Heparin/Chitosan B Mixtures Using Toluidine Blue Assay

In order to investigate the effect of chitosan B in a heparin solution using toluidine blue assay, a series of chitosan B solutions with increasing amounts of chitosan B were added to a fixed concentration heparin solution. The UV absorption of heparin with and without added chitosan B at 629 nm were measured using toluidine blue assay. Results are given in Table 3. Heparin contains esterified sulfuric acid and reacts with aqueous toluidine blue solution. The color of the dye solution changed immediately from blue to red-violet. If the mixture was shaken with an immiscible organic solvent such as hexane, the heparin-dye complex was removed by adsorption at the interface, while the uncombined dye remained in the aqueous phase and retained its normal color. A decrease in absorbance of aqueous toluidine blue solution at 629 nm indicates an increase of the amount of heparin. After adding the chitosan B to the heparin solution, it was found no heparin-dye complex occurred in the organic solvent. The absorptions of the aqueous layer were found to be out of range of the calibration curve that had been prepared. It is assumed that the sulfate groups of heparin react first with chitosan B such as hydroxyl or amine group, there was no heparin-toluidine blue complex formed in the hexane solvent. This suggests that the toluidine blue assay is not suitable for the determination of heparin concentration with chitosan B.

TABLE 3
The UV absorption of toluidine blue with and without chitosan B

| Heparin (μg) | Chitosan B (μg) | Abs |
|---|---|---|
| 51.96 | 0 | 0.141 |
| 51.96 | 20 | 0.476 |
| 51.96 | 40 | 0.466 |
| 51.96 | 60 | 0.450 |
| 51.96 | 80 | 0.439 |
| 51.96 | 100 | 0.428 |

### Electrochemical Properties

Dispersions were cast onto stainless steel coupons for electrochemical characterization. All of the cast films on stainless steel coupons exhibited poor adhesion with the films peeling off when immersed into the electrolyte. UV treatment (5, 10 and 20 min) of the coupons was performed in an attempt to improve adhesion. After 20 min UV treatment, the adhesion of the cast films was greatly improved. However, only the SWNT-DNA film remained intact after immersion, with the SWNT-Dextran and SWNT-Heparin films partially dissolving.

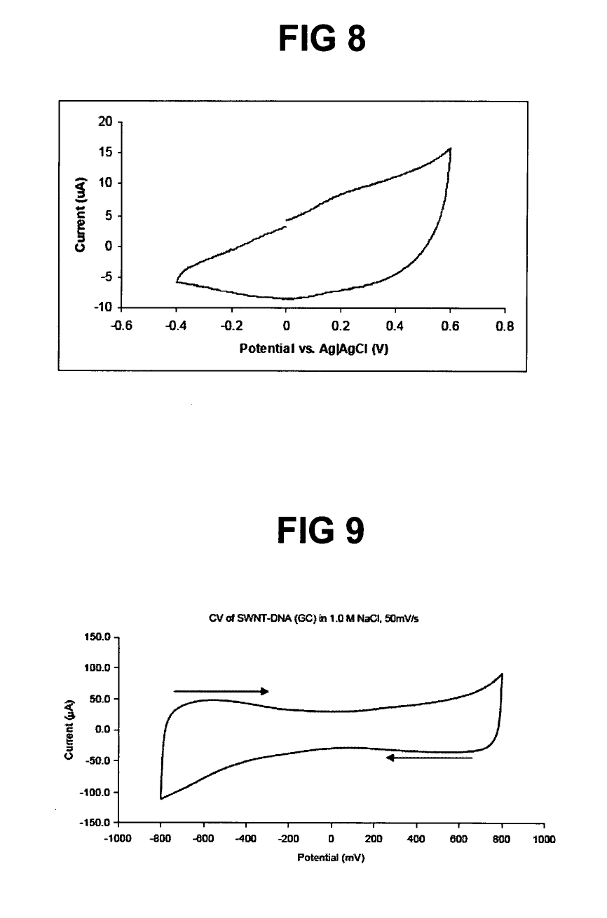

This led to a further study to characterize CNT-biomolecule coatings on glassy carbon. The cyclic voltammograms obtained on GC were typical of those observed for carbon nanotube electrodes previously with a large capacitative component obvious. The redox couple a and a′ of FIG. 7 is attributed to the oxidation and reduction of the Fe catalyst present in the SWNT source. This redox couple was not observed in cyclic voltammetry (CV) readings obtained for DWNT-DNA films on glassy carbon electrodes (FIG. 8), indicating that the DWNT preparation had lower levels of Fe catalyst contaminant. FIG. 9 is a graphical representation of a cyclic voltammogram obtained for SWNT-DNA (40 μg) cast on 0.07 cm₂ GC electrode in 1.0 M NaCl.

It was necessary to reduce the potential range in the CVs recorded for the stainless steel coupons. When the stainless steel coupon was scanned between −800 mV and +800 mV corrosion behavior was observed in the CV. The electrolyte solution turned yellow/red, possibly the result of iron leaching from the coupon.

### Preliminary Cell Culture Experiments

As indicated above, DWNT dispersions were shown by cyclic voltammetry (CV) to have lower levels of Fe contaminant than that present in SWNT dispersions. DWNT-biomolecule dispersions were used as substrates for preliminary cell culture experiments. DWNT-chitosan (DWNT/CH), DNA (DWNT/DNA) or hyaluronic acid (DWNT/HA) coatings prepared from 0.5%/0.5% dispersions in water were drop cast into 12-well polystyrene or 96-well polypropylene plates and dried overnight before soaking in cell culture media overnight. The coatings were washed twice in water and sterilized by drying from 70% ethanol under UV light. DWNT/chitosan coatings remained intact, whereas DWNT/DNA and DWNT/HA lifted from the plastic substrate and partially dissolved. L929 (mouse fibroblast) cells were cultured on these coatings and were found to grow well with normal adherent morphology.

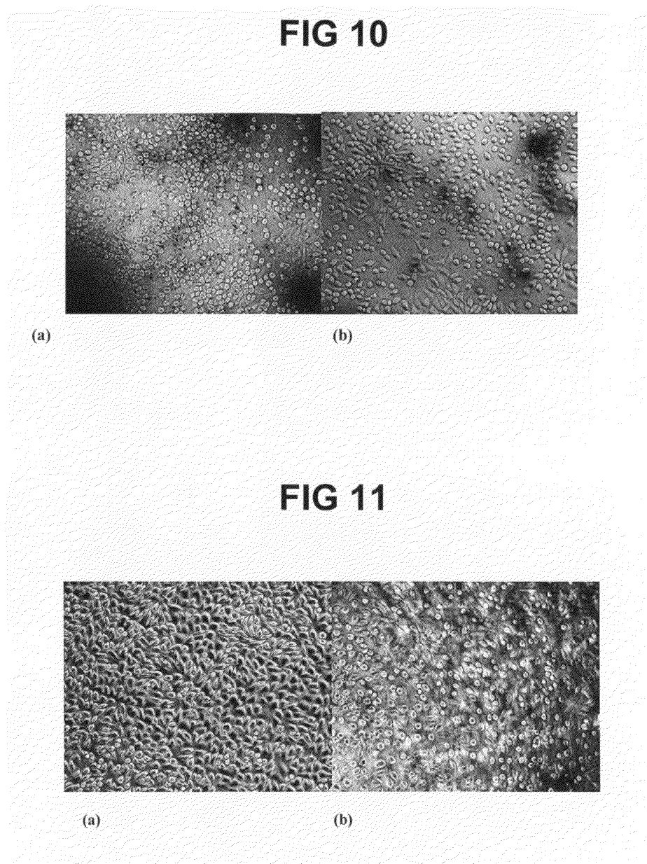

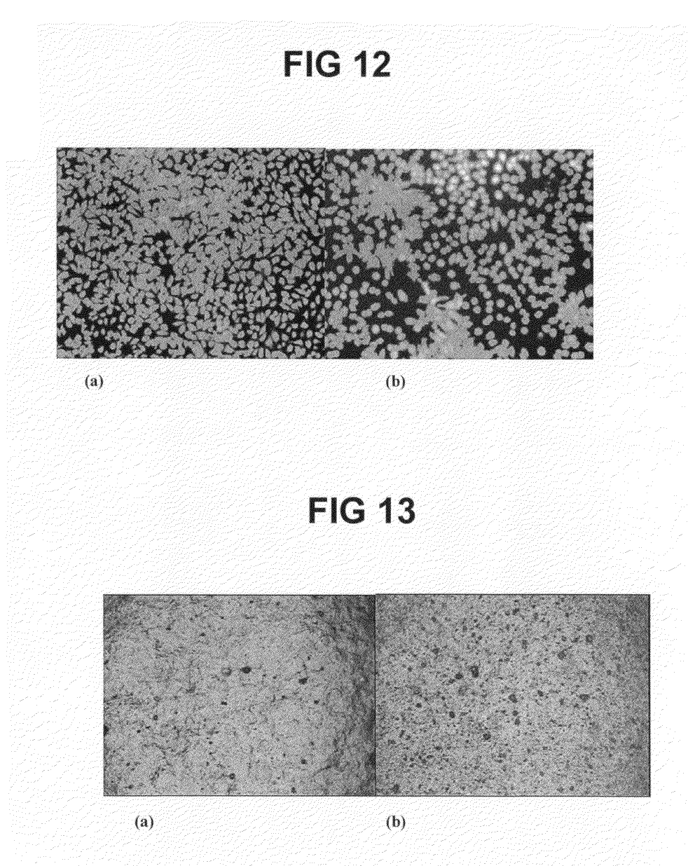

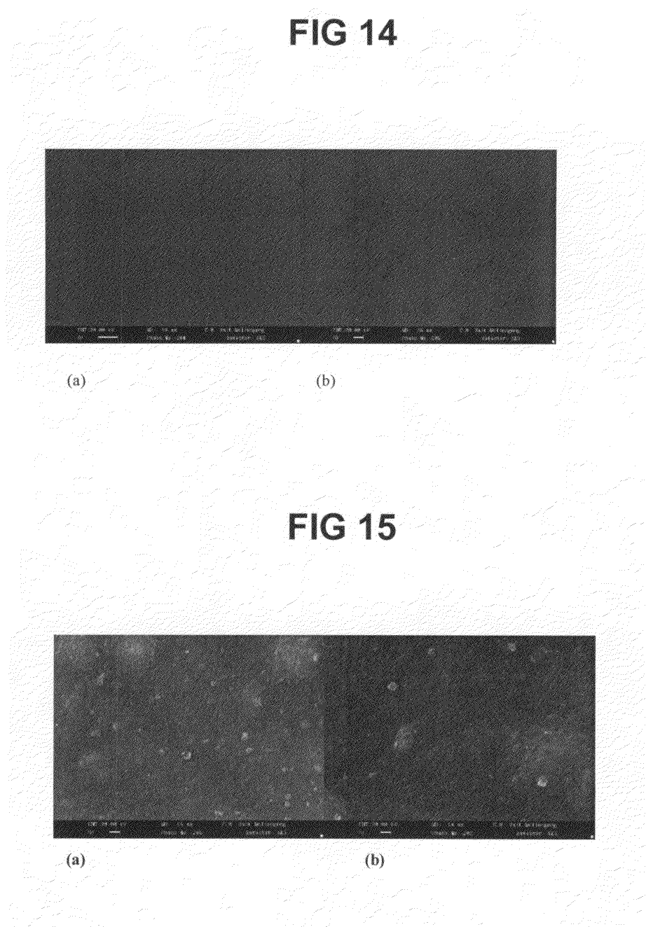

Confluent cultures were obtained by 72 hours, with the best growth occurring on DWNT/chitosan coatings (FIG. 10). The presence of metabolically active cells on all three coatings was evident by the observed increase in cell number during the 3 days of culture and by the presence of brightly fluorescent calcein AM-stained cells (FIG. 10). Calcein AM enters cells and is cleaved to form a bright green fluorescent product in the presence of intracellular esterases, indicating the presence of metabolically active cells. FIG. 10(a) shows cells cultured on DWNT/Chitosan coating on polypropylene and FIG. 10(b) shows the same coating on polystyrene. FIG. 11 shows fluorescence images of L929 cells cultured on (a) DWNT/DNA/polystyrene and on (b) DWNT/HA/polypropylene. FIG. 12 shows fluorescence images of calcein-stained L929 cells cultured on (a) DWNT/DNA and on (b) DWNT/CH coating on polystyrene.

### EXAMPLE 2

### Preparation and Characterization of CNT/SIBS Dispersions and Films

Poly(styrene-β-isobutylene-β-styrene (SIBS) has proven to be an effective biomaterial for coating stents. Paclitaxel can be integrated throughout SIBS to provide an effective “controlled” release system, minimizing the risk of restenosis. Ranade, S. V., Miller, K. M., Richard, R. E., Chan, A. K., Allen, M. J., Helmus, M. N., J. Biomed. Mater. Res. 2004, 71A, 625-634. Based upon this knowledge, a protocol was developed to form stable conducting CNT/SIBS coatings for stents in order to determine the effect of carbon nanotubes on cell adhesion and proliferation. The effect of solvent type, sonication conditions, and method of film preparation on the visual quality of SWNT/SIBS films and on the conductivity, as measured by four point probe, was investigated.

### Preparation of CNT Dispersions

Initially a range of organic solvents including toluene, cyclohexane, chloroform and tetrahydrofuran (THF) were used to dissolve SIBS and to disperse SWNTs. A variety of sonication conditions were investigated and the films produced from the resulting dispersions were inspected by light microscopy and characterized by 4 point probe conductivity. Toluene was found to be the best dispersant for SWNTs in terms of the quality and conductivity of the drop cast dispersions. Sonication times were increased from 15 to 30 min and power levels of 30 and 35% power tested. The variation in sonication conditions resulted in production of better SWNT dispersions, producing films with approximately a 3-fold increase in conductivity attributed to longer sonication times, and a slight increase in conductivity only attributed to the increase in power. The optimum sonication conditions that were maintained for this study were 45 mins of pulsed sonication (2 secs on, 1 sec off) at 35% power using a solid probe tip.

To improve the quality of dispersions, SWNTs were also dispersed in toluene containing 5% SIBS. However, conductivities were lower when SIBS was included in SWNT dispersions than when SIBS was pre-cast as a separate layer. Optical micrograph images of 0.15% SWNT/5% SIBS single layer (FIG. 13(a)) and 2-layer (FIG. 13(b)) coatings showed that SWNTs were well dispersed, with few occlusions of non-dispersed tubes.

There were fewer occlusions where SIBS was included as a dispersant (in single layer films) indicating that the dispersion was assisted by SIBS. For SWNTs alone, drop dispersions were not as even (not shown), with thick and thin areas clearly visible, indicating that in the absence of SIBS, CNTs had coalesced upon drying of the films. This illustrated the improvements in coatings made in the presence of SIBS, either when incorporated in the dispersion, or when used as a base for casting films.

### Film Formation

Two methods were used to form the SWNT/SIBS films: 1) 2-layer approach where a layer of SIBS is drop cast and allowed to dry, followed by the application of a SWNT dispersion (prepared from the same solvent as the initial SIBS layer); and 2) 1-layer approach where the SWNT is dispersed in a solution of SIBS in solvent and a film is cast from the resulting SWNT/SIBS dispersion.

### Addition of Cationic Surfactant Tetrahexadecyl Ammonium Bromide (“THAB”)

In an attempt to decrease the degree of SWNT aggregation and hence improve the conductivity of cast dispersions, the effect of the surfactant THAB on SWNT dispersions was investigated. THAB was added to SWNT/toluene dispersions with or without the addition of SIBS at a THAB:SWNT ratio of either 0.1% w/w or 1% w/w and sonicated. The use of THAB produced improved dispersions with a 20% increase in conductivity over that of the corresponding film with no surfactant present.

### Film Characterization

### Four-Point Probe Conductivity

The general trend in conductivity of the films produced from the two methods indicated that the 2-layer method produced films with conductivity up to 5 times higher. While not wishing to be bound by theory, the mechanism of film formation for the 2-layer method may involve the solvent of the SWNT dispersion partially or wholly dissolving the underlying SIBS layer and forming a composite 3-dimensional SWNT/SIBS film. The lower conductivity of films cast using the single-layer method may indicate that SIBS is coating the SWNTs during the dispersion process. The lower conductivity of films produced by this method may be attributed to a more complete coating of SWNTs by non-conducting SIBS, when compared with the 2-layer protocol.

The conductivity of SWNT/SIBS films was improved by increasing the nominal SWNT content from 0.05% w/v to 0.30%. The presence of a precast SIBS layer, in 2-layer films, assisted the casting of SWNT dispersions. Mixed SWNT and SIBS single layer films were easier to cast and gave more even and more finely dispersed films than for corresponding 2-layer films. Conductivity of SWNT films cast in the absence of SIBS was dependent on the concentration of incorporated SWNTs up to a limiting value of 3.9×10−2 S/sq for 0.20% or greater SWNT dispersions. At lower concentrations of SWNTs, the conductivity of 2-layer SWNT on SIBS films was an order of magnitude lower than for the corresponding dispersion in the absence of SWNTs.

However, the conductivity of 2-layer films continued to increase with increasing incorporation of SWNTs such that conductivity of 2-layer films containing (nominally) 0.30% SWNTs was increased to one third that of the corresponding film cast without SIBS, being measured at 1.1×10−2 S/sq. Conductivity of single layer mixed SWNT/SIBS films also increased with the concentration of nanotubes. Conductivity for these films was an order of magnitude lower than for the corresponding 2-layer films, reaching a maximum of 1.6×10−3 S/sq for (nominally) 0.30% SWNTs.

### Functionalized SWNT Films

The use of carboxylated SWNTs for film preparation was investigated and successfully prepared. However, in preliminary studies, the conductivity of these films was at least an order of magnitude lower than for the corresponding non-functionalized films. Thus, for application in which electrically conductive composite regions are desired, for example for purposes of electromechanical actuation, the carboxylated SWNTs may not, without further modification, possess a threshold level of electrical conductivity.

### Preparation of DWNT and Very Thin MWNT Films

When DWNTs and very thin MWNTs were dispersed in toluene under the same conditions as for SWNT dispersions, there was a much lower incorporation of tubes into dispersions. For a nominal 0.25% dispersion, there were only 51% of DWNTs and 60% of MWNTs incorporated, in contrast to 80% in the case of SWNTs. The conductivity of nominally 0.25% SWNT films produced either as a single layer or as 2-layer films on SIBS, was 3 times higher for SWNTs than for DWNT and MWNTs, directly reflecting the actual concentration of nanotubes incorporated. The conductivity of 2-layer films of approximately the same actual incorporated nanotubes was very similar for SWNTs, DWNTs and MWNTs.

### Characterization of SWNT/SIBS Films by Light Microscopy, FESEM and SEM

The addition of 0.15% SWNTs did not affect the surface morphology of 5% SIBS films, as seen by SEM (FIGS. 13(a)-13(b)). These single layer films (FIG. 13(a)) produced from mixed SIBS and SWNTs and cast onto stainless steel were very even, but contained surface cracks. For 2-layer films (FIG. 13(b)), the more uneven surface morphology was defined by the presence of SWNTs, in that the cauliflower-like appearance was unaffected by the base layer of SIBS.

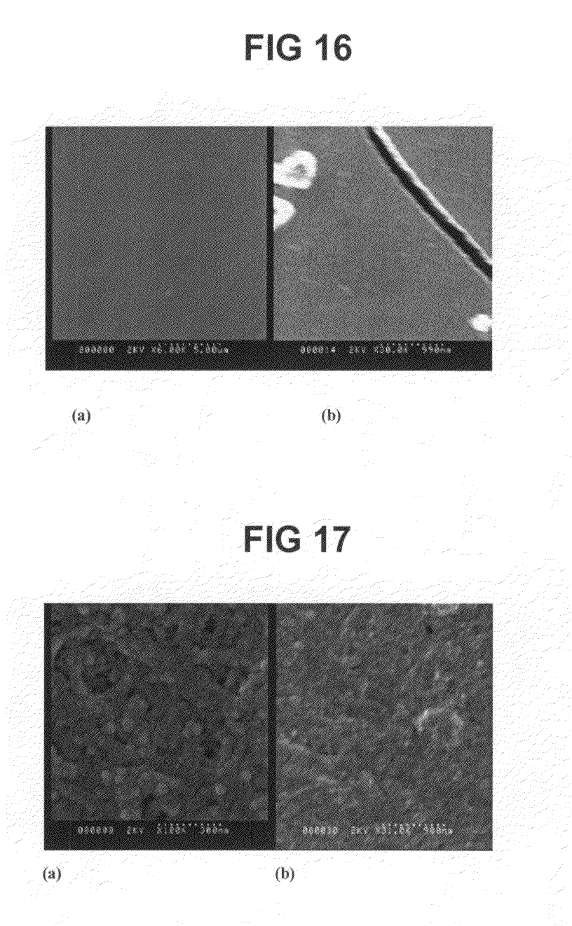

Field emission scanning electron microscopy (FESEM) confirmed that at higher resolution, SIBS only films were very even and smooth, with no distinctive morphological details (FIG. 16(a)). The smooth appearance of SIBS coatings was evident at lower concentrations of SWNTs (0.15%) in mixed single layer coatings (FIG. 16(b)). However, at higher concentrations, the coating resembled the matted network of coatings prepared in the absence of SIBS (FIGS. 17(a)-(b)). This may indicate that there was insufficient SIBS to completely coat the nanotubes at 0.25% SWNTs (not shown). Formation of a preformed SIBS layer, prior to layering with SWNTs in 2-layer films, assists in dispersing the tubes evenly. At the high resolution of FESEM, this presented as a more even appearance of the film, without the “cauliflower-like” globular formations that were present in SWNT-only films (FIG. 17(b)).

### Electrochemistry

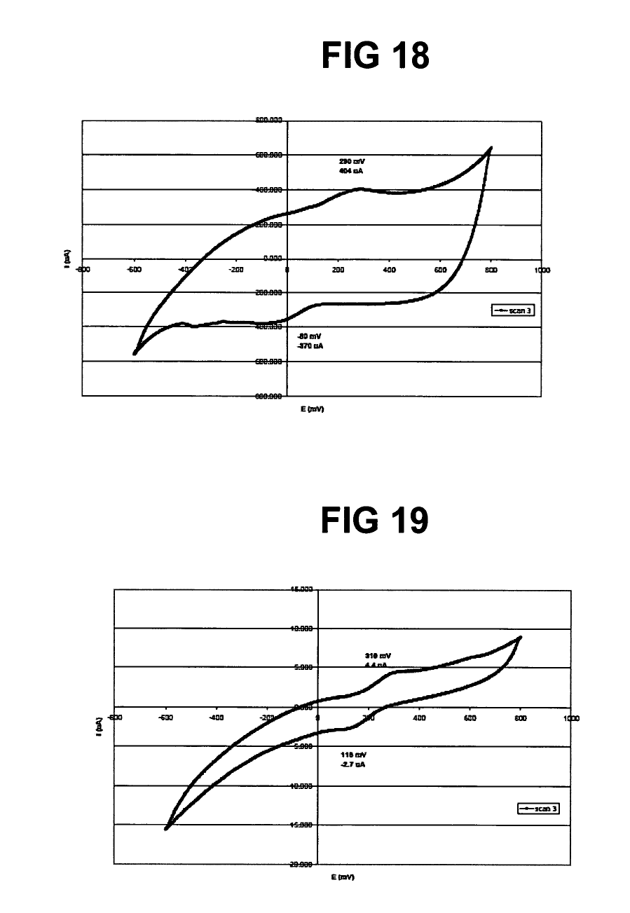

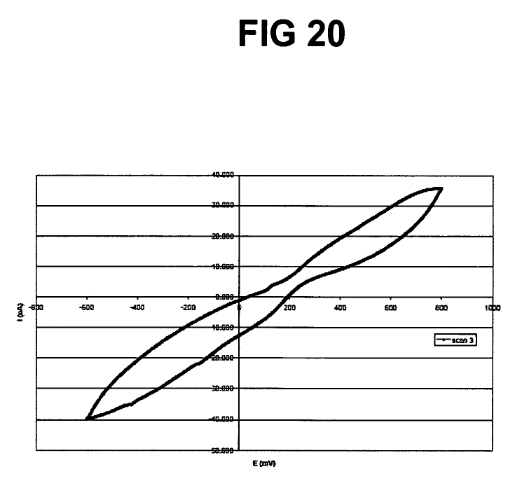

The capacitance and conductivity of prepared films on ITO glass and on non-conductive glass was characterized by cyclic voltammetry (CV) in phosphate buffer solution (PBS) and in phosphate buffer containing 1 mM K₃Fe(CN)₆ (“potassium ferricyanate”). On glass, only films cast from SWNTs alone were conductive enough to allow reasonable CVs to be obtained. Ferricyanide redox peaks were visible but were masked by the high background currents typical of these high capacitance coatings (FIG. 18). Current flows were lower on glass than on ITO-coated glass indicating that part of the measured capacitance on ITO-glass was due the substrate. Current flow in K₃Fe(CN)₆ was lower and peaks were further separated on glass than on ITO glass (370 mV cf 123 mV), again indicating the porosity of the films. There was little difference between CVs of single layer SWNT/SIBS films on glass and on ITO-glass in ferricyanide, suggesting that these films are not porous. These films were electroactive enough to allow ferricyanide redox peaks to be observed, but current flows were very low (FIG. 19). For 2-layer SWNT on SIBS films, low current flows, the angle of the CV and the lack of redox peaks suggested a resistive film (FIG. 20). For SIBS only films, there was a low conductivity on ITO-glass whereas there was no current flow on standard glass (not shown), indicating the porosity of SIBS films, as indicated by the surface cracks visible by SEM.

### Electrochemistry of 0.25% SWNT Films

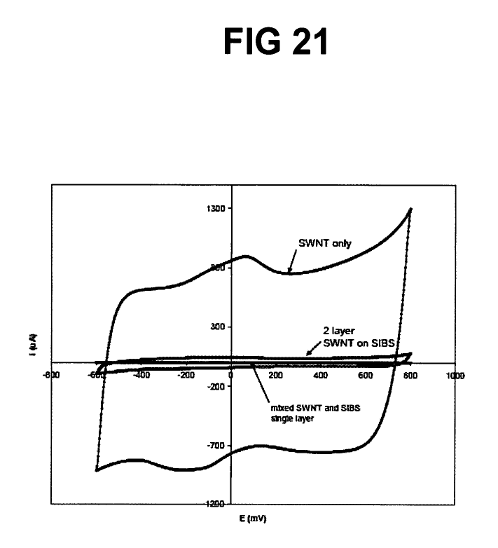

CVs were also obtained for films prepared from 0.25% SWNT films on ITO glass in phosphate buffer and in 1 mM K₃Fe(CN)₆. CV of SWNTs alone in phosphate buffer was typical of that of a high surface electrode with current flows at 0 mV of around +/−700 uA, which were higher than for the corresponding 0.15% SWNT films (+/−500 μA) indicating increased surface area and/or conductivity that arises from increased SWNT content. As for 0.15% SNWT films, ferricyanide redox peaks were visible due to the high background current of these films. Current flows were much lower in the presence of SIBS, at only +0.38/+0.80 μA for a mixed 0.25% SWNT/SIBS single layer film (FIG. 21), indicating the relatively low conductivity of these films. Current flows were also low in 1 mM K₃Fe(CN)₆ and no redox peaks were visible. For 2-layer SWNT on SIBS films, current flow at 0 mV was around +/−35 μA, indicating an improvement in conductivity over the single layer films, however conductivity was very low compared to films produced in the absence of SIBS. Ferricyanide redox peaks for this film were visible but very broad.

### Electrochemical Stability

Scanning of 2 layer SWNT/SIBS films on ITO glass for 150 cycles between −400 mV and +800 mV at 5 mV/sec in PBS showed the films to be electrochemically stable in that there was no change in capacitance of the films over 150 cycles.

### Cell Culture

The suitability of SWNT/SIBS coating for cell growth was assessed using the mouse fibroblast cell line NCTC929 (L929). L929 cells are cultured in DMEM:F12 media containing 5% FCS. Cells were cultured at 37° C., in a humidified, 5% CO2 atmosphere. L929 cells were trypsinised and split 2-3 times weekly. Calcein loading of L929 cells consisted of 5 μM Calcein AM being added to cells in a standard culture medium and incubated for at least 15 mins at 37° C., before visualizing with an inverted fluorescence microscope.

### Substrates for Growth of L929 Cells on SWNT/SIBS Coatings

Initially SWNT/SIBS coatings were prepared on glass cover slips and placed into 12-well polystyrene (PS) plates for cell growth experiments. Due to problems in quantitation caused by cells growing around the cover slips, on the preferred PS surface, coatings were prepared directly in polypropylene (PP) 96-well plates which were resistant to the solvent (toluene). The range of coatings being investigated has recently been expanded to include aqueous dispersions produced from DWNTs and the biomolecules: DNA, hyaluronic acid and chitosan. These coatings are compatible with standard PS tissue cultureware. However, the adhesion of DWNT/DNA and DWNT/HA coatings to PS is poor.

### Growth of L929 Cells on SWNT/SIBS Coatings

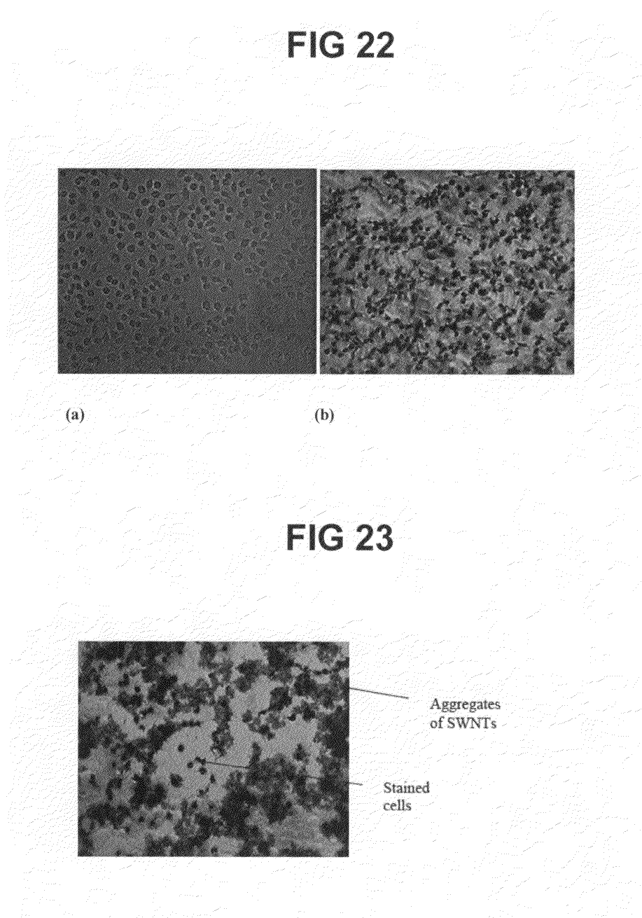

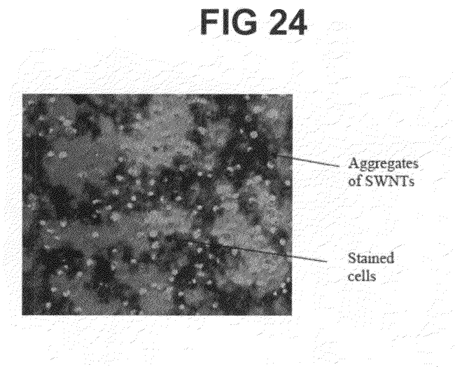

Preliminary cell growth experiments showed that L929 cells grew on a range of SWNT/SIBS coatings, including single layer and 2-layer films, or on SIBS only. Cells were characterized by phase contrast microscopy, before and after staining with MTT reagent. L929 cells grew well on SIBS coatings and were metabolically active, as evidenced by MTT staining (see FIGS. 22(a)-(b)). However, on SWNT coatings, cells did not attach as well as on polystyrene or on SIBS, maintaining a rounded morphology. The cells were, however, metabolically active on SWNT coatings, as evidenced by MTT staining (FIG. 23) and observations made of calcein-stained cells (FIG. 24). Calcein AM (Molecular Probes) permeates cells and is cleaved by non-specific esterases within the cell to yield a green fluorescent product that can be characterized by fluorescence or confocal microscopy. The range of coatings has been expanded to include aqueous dispersions produced from DWNTs and the biomolecules: DNA, hyaluronic acid and chitosan.

### Quantitation of Cell Growth Using MTT and MTS Assays

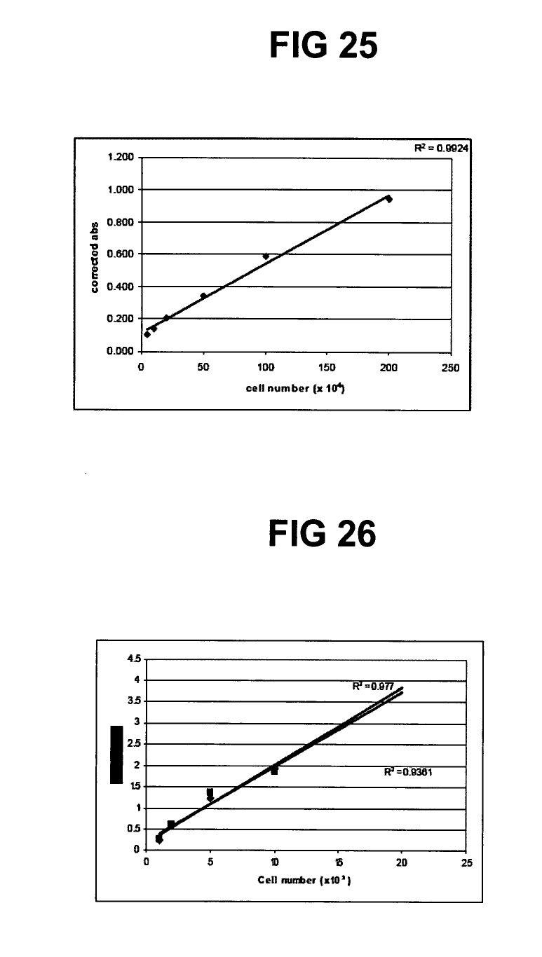

Calibration curves were obtained for both MTT [3-(4,5-dimethylthiazol-2-yl)-2,5-diphenyltetrazolium bromide] and MTS [3-(4,5-dimethylthiazol-2-yl)-5-(3-carboxymethoxyphenyl)-2-(4-sulfophenyl)-2H-tetrazolium] assays, both for cells plated out for 3 hrs only (actual cell number) and for cells plated out and allowed to proliferate for 48 h, i.e., under the same conditions that were used to test cell growth on SWNT/SIBS coatings, as discussed above. It was found that the MTT assay gave a linear relationship between cell number and corrected absorbance in the range of 5×10⁴ to 2×10⁶ cells (FIG. 25) in 12-well format, and MTS assay gave linear absorbance in the range 1-10×103 cells in 96-well format (FIG. 26). For the MTT assay, MTT reagent in PBS (Sigma-Aldrich) was added to cell cultures to give a final concentration of 0.5 mg/mL substrate. Cells were incubated under standard culture conditions for 4 hrs to allow product development. 50% media was removed and replaced with 0.1N HCl in IPA and placed on a shaking platform form approximately 1 hr, with trituration, to dissolve the product. 200 μL of supernatant was transferred to a 96-well plate and absorbance read at 570 nm with background correction at 690 nm. For the MTS assay, 10% by volume of Cell Titer 96 Aqueous Cell proliferation assay solution (Promega) was added to cell culture wells and incubated for 4 h under standard cell culture conditions. 200 μL of each sample was transferred to a 96-well plate and absorbance read at 490 nm using a 96-well plate reader.

### MTT and MTS Assays on SWNT/SIBS Films

Initially, background staining due to SIBS and/or SWNT coatings was assessed in order to determine the feasibility of using these assays to quantitate cell growth on the coatings. Background staining was less significant using the MTS assay (Promega) than for the MTT assay (Sigma). MTS assay was therefore used to quantitate cell growth in all further experiments. An initial experiment was done to compare L929 cell growth on glass cover slips coated with SIBS and/or SWNT coatings and placed into the wells of a 12-well tissue culture plate. In this experiment, 5×10⁴ cells were seeded per 12-well plate well. However, many cells grew around the margins of the wells rather than on the cover slips. Cover slips were removed and transferred to fresh wells for MTT quantitation. Therefore, the results gave only a relative measure of differences in cell growth between the different coatings.

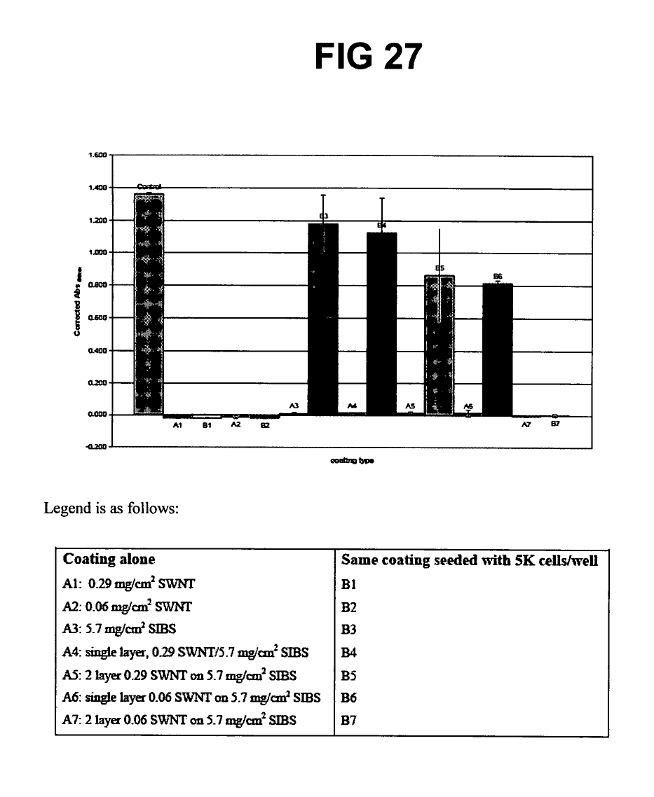

Results of the MTT assays for cells growing on the coatings suggested that cell growth was better on single layer coatings than on 2-layer coatings. However, absorbance levels were only in the range of those obtained for no-cell control coatings due to the loss of cells as described above. This cell growth assay was improved by casting SWNT dispersions directly into 96-well tissue culture trays and using the MTS assay, which gave lower background absorbance, rather than the MTT assay. The sensitivity of the assays was improved by increasing incubation times for cells on the coating to 72 h. Due to the incompatibility of toluene with standard PS tissue culture plastic, 96-well polypropylene (PP) plates were used to quantitate cell growth on drop cast coatings. MTS assays on coatings formed on PP plates confirmed that cell growth was better on single layer coatings than when a SIBS layer was laid down first (2 layer coating) (FIG. 27). Cell growth was poor on SWNT-only coatings but was improved in the presence of SIBS, either as a SIBS-only coating or as mixed SIBS/SWNT coatings. Cell growth on SIBS alone was only marginally less than that on PP itself. Assays were performed in triplicate. The control lane consisted of 5,000 cells seeded per well on a polypropylene plate. Cell morphology was altered on PP, whether coated or uncoated. L929 cells had a more rounded morphology on PP and on all of the SIBS and SWNT/SIBS coatings, than the characteristic “flattened” morphology that is typical of L929 cells growing on tissue culture-treated PS cell culture surfaces.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavor to which this specification pertains.

Although various embodiments are specifically illustrated and described herein, it will be appreciated that modifications and variations of the present invention are covered by the above teachings and are within the purview of the appended claims without departing from the spirit and intended scope of the invention.

1. A medical device comprising at least one composite region, said composite region comprising carbon particles and a polymer comprising a biocompatible copolymer comprising a block copolymer comprising a polyisobutylene block and a polystyrene block.

2. The medical device of claim 1, wherein the composite region comprises at least one composite carrier region and a therapeutic agent disposed within said composite carrier region.

3. The medical device of claim 2, wherein the composite region comprises at least one composite barrier region disposed over all or a portion of said device.

4. The medical device of claim 1, wherein said therapeutic agent is an anti-proliferative agent comprising paclitaxel.

5. The medical device of claim 1, wherein the carbon particles comprises molecular carbon made of carbon atoms that are predominantly in a sp² hybridized form.

6. The medical device of claim 1, wherein the carbon particles are selected from the group consisting of graphite, fullerenes and carbon nanotubes comprising single-wall carbon nanotubes or functionalized carbon nanotubes and the polymer comprises a styrene-isobutylene block copolymer.

7. The medical device of claim 1, wherein said composite region comprises two or more layers with at least one layer comprising a polymer and at least one layer comprising carbon particles.

8. The medical device of claim 1, wherein said composite region comprises a first layer comprising a polymer and a second layer comprising carbon particles.

9. The medical device of claim 8, wherein said first and second layers each have a surface and at least a portion of each of the surfaces are bonded to each other by application of heat, pressure, or an adhesive.

10. The medical device of claim 8, wherein the first layer comprises a polymer having a surface at least a portion of which surface is attached to the second layer by applying the surface with a solution comprising the polymer dissolved in a solvent, wherein the polymer comprises a styrene-isobutylene copolymer and the solvent comprises toluene.

11. The medical device of claim 8, wherein the therapeutic agent is disposed within at least one of the first layer or the second layer of the composite region.

12. The medical device of claim 8, wherein the therapeutic agent comprises biologically active molecules that are embedded within at least one of the first layer or the second layer.

13. The medical device of claim 8, wherein said medical device comprises a stent having two ends and an interior surface and an exterior surface and either the first or second layer is disposed on at least a portion of the interior surface of the stent and either the first or second layer is disposed on at least a portion of the exterior surface.

14. The device of claim 13, wherein the first layer covers the entire exterior surface of the stent and the second layer covers the entire interior surface of the stent, wherein said first and second layers each have a surface and at least a portion of each of these surfaces are bonded to each other by application of heat, pressure, or an adhesive adjacent to the ends of the stent.

15. The medical device of claim 8, wherein the second layer comprising carbon particles is a film formed from a dispersion comprising carbon nanotubes, a solvent, and a surfactant.

16. The medical device of claim 1, wherein said composite region is a conductive region.

17. The medical device of claim 8, wherein the second layer comprising carbon particles is a porous film.

18. The medical device of claim 8, wherein the second layer comprising carbon particles is a film comprising styrene-isobutylene copolymer that is continuous or perforated with holes.

19. The medical device of claim 1, wherein said medical device is selected from a balloon, a guide wire, a vena cava filter, a stent, a stent graft, a vascular graft, a cerebral aneurysm filler coil, a myocardial plug, a heart valve, a vascular valve, and a tissue engineering scaffold.

20. A composite material for use in an insertable or implantable medical device comprising a composite region, said composite region at least one layer of carbon particles disposed over all or a portion of the device and at least one layer of a polymer disposed over all or a portion of the device, wherein the polymer comprises a styrene-isobutylene copolymer, wherein a therapeutic agent is disposed within the polymer.
